(19) United States
(12) Patent Application Publication
(10) Pub. No.: US 2023/0014138 A1
(43) Pub. Date: Jan. 19, 2023

(54) NEW COSMETIC COMPOSITIONS

(71) Applicant: YODI SAS, PARIS (FR)

(72) Inventor: Hélène AZANCOT, NEUILLY SUR SEINE (FR)

(73) Assignee: YODI SAS, PARIS (FR)

(21) Appl. No.: 17/782,432

(22) PCT Filed: Dec. 2, 2020
(86) PCT No.: PCT/EP2020/084318

§ 371 Date: Jun. 3, 2022

(30) Foreign Application Priority Data
Dec. 4, 2019 (FR) FR1913768
Jun. 9, 2020 (FR) FR2006002

(51) Int. Cl. A61K 8/60; A61K 8/34; A61K 8/49; A61K 8/92; A61K 8/9794; A61K 8/73; A61Q 5/02; A61K 8/9789; A61K 8/19; A61K 8/99; A61Q 19/10

(52) U.S. Cl. CPC A61K 8/60; A61K 8/345; A61K 8/4926; A61K 8/922; A61K 8/9794; A61K 8/732; A61Q 5/02; A61K 8/9789; A61K 8/4913; A61K 8/19; A61K 8/99; A61Q 19/10

(57) ABSTRACT

New cosmetic compositions and their use, in particular, as a shampoo or a cleanser. The compositions are in powder form substantially free of water. The compositions are composed of constituents and ingredients that are natural or of natural origin, non-allergenic, healthy, and free of preservatives. Also, a device including the novel compositions.

The present invention relates to new cosmetic compositions and their use, in particular as shampoo or cleanser. The present invention also relates to a device comprising the novel compositions.

### BACKGROUND OF THE INVENTION

Most personal hygiene products, such as shampoos or cleansers, are marketed in liquid form. These products mainly comprise water, sometimes up to 90% by mass relative to the total weight of the product. The presence of large quantities of water has a negative impact on several points, for example on the environment or on the requirements related to the transport of the product. Indeed, the high volume of packaging leads to a high cost of transport by the distributor and constraints for the consumer, for example during a trip.

Given the presence of water, cosmetic compositions in liquid form require preservatives, and have quantities of active substances in a reduced percentage. As a result, the consumer spends a large sum for few active substances.

In recent years, a few shampoos and cleansers in powder form have appeared on the market. However, most of these products contain chemical synthetic ingredients, potentially irritating and/or allergens. In addition, the diversity of hair and skin types means that the range of products available does not meet the specific needs of each consumer.

There is therefore a need to provide alternative compositions, comprising ingredients that are natural or of natural origin, non-allergenic, healthy, and free of preservatives, preferably having a protective activity for the skin flora.

One of the aims of the invention is to provide a new combination in powder form, comprising a prebiotic.

Another aim of the invention is a novel cosmetic composition in powder form comprising a prebiotic.

A third aim of the invention is to be able to use the new combination, or a composition comprising it, as a shampoo or cleanser.

A fourth aim is to provide a device comprising a shampoo or cleanser in powder form, which is easy to use.

One of the aims is to provide a cosmetic composition free of preservatives.

One of the other aims is to provide a cosmetic composition in which more than 15% by mass relative to the total weight of the composition, in particular more than 17%, consists of constituents and ingredients having a beneficial property for the skin.

The inventors have begun in-depth research in order to develop shampoos and powder cleansers, having interesting sensory properties, in particular with respect to foaming power and rinsability. Developed powders are fine and soft, and produce a smooth feel when handling.

A first object of the present invention is a combination comprising the following 5 constituents:
a powder adhesion agent,
mannitol,
α-glucan oligosaccharide,
at least one oil, and
at least one surfactant,

said combination being in powder form substantially free of water,

said adhesion agent, said mannitol, said α-glucan oligosaccharide, and said at least one surfactant being in powder form, said at least one oil being in particular in powder form,

wherein said combination optionally further comprises at least one ingredient.

The sum of the constituents and ingredients in powder form represents in particular from 90% to 100% by mass relative to the total weight of the combination according to the present invention.

With respect to the sum of the constituents and ingredients, the term “from 90 to 100%” also means the following ranges: from 92 to 100%, from 94 to 100%, from 96 to 100%, from 98 to 100%, from 90-98%, 90-96%, 90-94%, 90-92%, 91-99%, 92-98%, 93-97%, 94-96%.

According to a particular embodiment, the present invention relates to a combination as defined above, comprising the following 5 constituents:
a powder adhesion agent,
mannitol,
α-glucan oligosaccharide,
at least one oil, and
at least one surfactant,

in which the mass percentage of water relative to the total mass of the combination is between 0 and 10%, in particular from 0.1 to 10%, in particular from 0.1 to 5%,

said combination being in powder form,

said adhesion agent, said mannitol, said α-glucan oligosaccharide, and said at least one surfactant being in powder form, said at least one oil being in particular in powder form,

wherein said combination further comprises at least one ingredient,

the total weight of said at least one ingredient is in particular comprised from 0 to 15% by mass relative to the total weight of the combination, in particular from 0 to 10%, or from 2 to 15%.

According to a particular embodiment, the present invention relates to a combination as defined above, in which the mass percentage of water relative to the total mass of the combination is between 0% and 1%, in particular from 0 to 0.5%, and the total weight of said at least one ingredient is between 2 and 15% by mass relative to the total weight of the combination.

According to a particular embodiment, the present invention relates to a combination as defined above, in which the mass percentage of water relative to the total mass of the combination is between 0.1 and 10%, in particular at least 1% to 10%, and the total weight of said at least one ingredient is 0% by mass relative to the total weight of the combination.

According to a particular embodiment, the present invention relates to a combination as defined above, in which the mass percentage of water relative to the total mass of the combination is between 0% and 1%, in particular from 0 to 0.5%, and the total weight of said at least one ingredient is 0% by mass relative to the total weight of the combination.

According to a particular embodiment, the present invention relates to a combination as defined above, in which the mass percentage of water relative to the total mass of the combination is 0.1 to 10%, in particular at least 1 to 10%, and the total weight of said at least one ingredient is between 2 and 15% by mass relative to the total weight of the combination.

According to the present invention, the term “constituents” means the 5 constituents stated above, namely: a powder adhesion agent, mannitol, α-glucan oligosaccharide, at least one oil, and at least one surfactant. At least 3 of the 5 constituents have activity on the skin.

The synergy between the five constituents gives rise to a combination whose use in a cosmetic composition confers interesting properties, in particular with respect to sensory characteristics. According to the present invention, by “ingredient” is meant the compounds other than the 5 constituents which may be optionally contained. The ingredients have either an activity on the skin, or properties at the sensorial level.

The combination according to the present invention consists of constituents and ingredients that are natural, or of natural origin, and of non-animal origin.

The expression “association” is synonymous with “combination”.

By “powder adhesion agent” is meant, within the meaning of the present invention, a support which allows good adhesion of the powders. It is an agent allowing the adhesion of the various constituents and ingredients of the powder, and modifying the rheology of the combination during its use. These are more particularly viscosity-modifying additives making it possible to modify the texture of the combination during its use. The powder adhesion agents that can be used in the present invention are in particular carbohydrate-based biopolymers.

By “α-glucan oligosaccharide” is meant an oligosaccharide consisting of a sequence of D-glucose units linked together by α-form glycosidic bonds. α-glucan oligosaccharides are synthesized by a transglucosylation reaction catalyzed by enzymes from the glucan-saccharase family. The α-glucan oligosaccharide that can be used in the context of the present invention is a “prebiotic”. Prebiotics are used to maintain a favorable environment to the development of endogenous skin flora. Prebiotics are in particular used to guide the metabolism of all of the skin flora in order to maintain a good balance of the latter, while not promoting the development of certain pathogenic bacteria.

In the context of the present invention, the α-glucan oligosaccharide having prebiotic properties preferably consists of 2 to 50 sugar units, in particular 2 to 10 sugar units.

Among the oligosaccharides that can be used and are commercially available, mention may be made of Bioecolia® (INCI: α-glucan oligosaccharide) or Ecoskin® (INCI: Alpha-glucan oligosaccharide, Polymnia sonchifolia root juice, Maltodextrin, Lactobacillus) from the company Solabia, or Biolin/P (INCI: Inulin (and) Alpha-Glucan Oligosaccharide) from the company Gobiotics.

The combination according to the present invention preferably comprises 1 or 2 oils.

The combination according to the present invention preferably comprises 1 or 2 surfactants.

By “substantially free of water” is meant that the mass percentage of water relative to the total mass of the combination is comprised from 0 to 10%, in particular from 0 to 5%, in particular from 0 to 3%, in particular from 0 to 2%, in particular from 0 to 1%, in particular from 0 to 0.5%.

The combination in powder form, as well as the adhesion agent, mannitol, α-glucan oligosaccharide, and the at least one surfactant are solid at room temperature.

The at least one oil is in particular solid at ambient temperature.

By “ambient temperature” is meant, according to the present invention, a temperature between 20 and 25° C.

The at least one ingredient is in particular an active principle, chosen in particular from: flower acid, Zinc PCA, a pigment, the coal, an antioxidant, in particular vitamin C, or one of its derivatives, powdered tea, including matcha green tea, fermented tea, powdered kombucha tea, centella asiatica, or madecassoside, sugars, in particular xylitol, rhamnose, or fructooligosaccharides, aloe vera, a protein, in particular pea protein, soy protein, wheat protein, or rice protein, sweet almond milk, or one or more of its components, in particular prunus dulcis, rice syrup or acacia fiber, a perfume, in particular an apricot almond perfume, ghassoul, white clay, especially kaolin, sodium hyaluronate, hyaluronic acid, lactic acid, vanilla macerate (INCI: Corylus avellana Seed Oil/Vanilla planifolia fruit), maltodextrin, bamboo powder, spirulina, phycocyanin, fermented mushrooms, guarana, caffeine, acerola, chamomile, Ayurvedic powders, vitamins, in particular vitamin B3, or vitamin B5, glycosphingolipids and glycolipids, for example from the Ceramosides™ HP brand (INCI: Glycosphingolipides, glycolipides), exopolysaccharides, and postbiotics, in particular lactobacillus ferment such as the Lactobiotyl® brand (INCI: Maltodextrin and Lactobacillus Ferment), pyroctolamine, piroctone olamine, a camellia seed extract (INCI: Camellia oleifera seed extract), a corn-flower extract (INCI: Centaurea cyanus flower extract), a chamomile flower extract (INCI Maltodextrin, Chamomilla Recutita Flower Extract), or a mixture of these,

said at least one ingredient is in particular solid at room temperature.

The total weight of said at least one ingredient is in particular comprised from 0 to 15% by mass relative to the total weight of the combination, in particular from 0 to 10%, or from 2 to 10%.

“Zinc PCA” is a zinc salt of pyrrolidone carboxylic acid, which is marketed for example under the name Zincidone®.

Among the pigments, mention may be made of Unicert violet K7025-J (INCI: CI 60730), comprising the benzene sulfonic acid 2-[(9,10-dihydro-4-hydroxy-9,10-dioxo-1-anthracenyl)amino]-5-methyl monosodium salt (CAS 4430-18-6) dye.

Among the exopolysaccharides we can mention:

the saccharide isomerate, for example from the Epidermist™ 4.0 brand (INCI: Aqua, Saccharide Isomerate), from the EPS Seamat™ brand (INCI: Aqua, Saccharide Isomerate), from the EPS Seapur® brand (INCI: Water, Phenethyl alcohol, Saccharide isomerate), from the EPS Seaglow® brand (INCI: Water, Saccharide isomerate, Sodium benzoate, Citric acid), the Actibiome brand from Codif (INCI: Glycerin, Water, Seawater, Laminaria digitata extract, Chlorella vulgaris extract, saccharide isomerate, phenethylalcohol),

Alteromonas ferment extract, for example from the EPS Seafill™ brand (INCI: Aqua, Alteromonas Ferment Extract),

or the exopolysaccharides of the brand, EPS Seawhite®.

Among the Ayurvedic powders we can mention: Shikakai powder (INCI: Acacia concinna fruit powder), Reetha powder (INCI: Sapindus mukorossi powder), sidr (INCI: Zizyphus jujuba leaf powder), brahmi powder (INCI: Bacopa Monnieri), Bhringaraj Powder (INCI: Eclipta alba powder), amla (INCI: Emblica officinalis fruit powder), Fenugreek powder (methi) (INCI: Trigonella foenum-graecum seed powder), Hibiscus powder (INCI: Hibiscus sabdariffa flower powder), Kapoor Kachli (INCI: Hedychium Spicatum), Tulsi powder (INCI: Ocimum sanctum leaf powder), kachur Sugandhi powder (INCI: Kaempferia galanga root powder), Neem powder (INCI: Azadirachta indica leaves powder), Nagarmotha powder (INCI: Cyperus rotundus root powder), and orange powder (INCI: Citrus aurantium peel powder).

As “antioxidant”, mention may be made, for example, of tocopherol (vitamin E) or ascorbic acid (vitamin C), or a derivative of vitamin C, in particular ascorbyl glucoside.

The perfumes used in the present invention are natural perfumes or perfumes of natural origin. These include a herb and tea fragrance, an apricot almond fragrance, or a coconut milk fragrance, which are for example marketed by the company Natflor.

The combination according to the present invention is in particular free of: preservatives, such as phenylethanol, parabens, polyethylene glycol (PEG), sulphates, talc, silicones, allergens, synthetic fragrances, and irritating active ingredients.

The combination according to the present invention is in particular free of mineral salts of heavy metals.

The combination according to the present invention is in particular free of non-natural materials, or of non-natural origin, and of animal origin.

The expression “free” means quantities by mass of less than 100 ppm, in particular less than 50 ppm, in particular less than 10 ppm.

Examples of preservatives include triclosan, triclocarban, phenylethanol, benzyl benzoate, methylisothiazolinone, zinc pyrithione, benzalkonium chloride, and benzyl alcohol. The combination according to the present invention is free of any chemical, non-natural preservative.

Among the parabens one can cite, by example, methyl paraben, propyl paraben, butyl paraben, and ethyl paraben.

By “polyethylene glycol” is meant a polymer consisting of ethylene glycol monomers which may have various molecular weights, mention may be made, by example, of PEG-1000 and PEG-2000.

Among the silicones, mention may be made, by example, of dimethicone, cyclomethicone, amodimethicone, ceteraryl methicone, dimethiconol, and stearyl dimethicone.

By “allergens” is meant substances known to be irritating or allergenic. By example, mention may be made of: α-Isomethyl ionone, amyl cinnamal, amylcinnamyl alcohol, anisic alcohol, benzyl alcohol, benzyl benzoate, benzyl cinnamate, benzyl salicylate, butylphenyl methylpropional, cinnamal, cinnamyl alcohol, citral, citronellol, coumarin, eugenol, farnesol, geraniol, hexyl cinnamal, hydroxycitronnellal, isoeugenol, limonene, linalool, and 2-methyl octynoate.

By “irritating active ingredients” is meant products presenting a risk of skin irritation. It is any active ingredient known to have an irritating property in the field of cosmetics. We can cite as example: sulphated surfactants, such as sodium lauryl sulphate or sodium coco sulphate, and guar hydroxypropyltrimoniumchloride.

According to another particular embodiment, the present invention relates to a combination consisting of the following 5 constituents:
a powder adhesion agent,
mannitol,
α-glucan oligosaccharide,
at least one oil, and
at least one surfactant,

said combination being in powder form substantially free of water,

said adhesion agent, said mannitol, said α-glucan oligosaccharide, and said at least one surfactant being in powder form, said at least one oil being in particular in powder form.

According to another particular embodiment, the present invention relates to a combination consisting of the following 5 constituents:
a powder adhesion agent,
mannitol,
α-glucan oligosaccharide,
at least one oil, and
at least one surfactant,

said combination being in powder form,

in which the mass percentage of water is between 0 and 10% relative to the total mass of the combination,

said adhesion agent, said mannitol, said α-glucan oligosaccharide, and said at least one surfactant being in powder form, said at least one oil being in particular in powder form.

In this particular embodiment, the combination is free of ingredients other than the 5 constituents indicated above.

According to another particular embodiment, the present invention relates to a combination as defined below, free of mannitol.

According to another particular embodiment, the present invention relates to a combination as defined above, in which the density of the powder is between 0.35 and 0.55 g/ml, in particular from 0.4 to 0, 5 g/ml.

Within the meaning of the present invention, the density corresponds to the “unpacked” density.

According to another particular embodiment, the present invention relates to a combination as defined above, in which the adhesion agent for the powders is chosen from: corn starch, acacia powder, maltodextrin powder, apple extract powder, in particular Vitacell CS5 Apple from JRS Rettenmaier (INCI Pyrus Malus (Apple) Fiber), tapioca powder, in particular Jojoba Silk Like Powder from Jojoba desert (INCI: Simmondsia Chinesis (Jojoba) Seed Oil, Tapioca Pure), rice powder, rice bran extract, and arrowroot powder or a mixture of these.

According to another particular embodiment, the present invention relates to a combination as defined above, in which the oil is chosen from: jojoba oil, sweet almond oil, bitter almond oil, Argan oil, sesame oil, camellia oil, safflower oil, moringa oil, Castor oil, coconut oil, apricot oil, linseed oil, pomegranate oil, grapeseed oil, prickly pear oils, marula oil, evening primrose oil, avocado oil, raspberry oil, borage oil, olive oil, neem oil, sunflower oil, walnut or hazelnut oil, amla oil, plum oil, coffee oil, hemp oil, a vanilla macerate, an oil powder, and a fermented oil, or a mixture of these.

By “oil powder” we mean oils that are initially liquid at room temperature, but which have been formulated to be solid at room temperature. The oils can for example be encapsulated to produce microcapsules in powder form. Among encapsulating agents, mention may be made of alginate, maltodextrin, arrowroot powder, or corn starch.

Among the oils capable of being formulated in the form of a powder, mention may be made of vegetable oils, in particular oils as defined above.

Mention may in particular be made of argan oil powder (argan oil coated with maltodextrin), jojoba powder, castor oil powder, sweet almond, or coconut oil powder.

By “fermented oil” is meant an oil which has undergone a fermentation process. Among the oils capable of being fermented, mention may be made of vegetable oils, in particular oils as defined above. Mention may in particular be made of fermented jojoba oil, fermented argan oil, fermented moringa oil, fermented sesame oil, fermented sweet almond oil, fermented hemp oil, fermented safflower oil, fermented camellia oil, or fermented olive oil.

According to another particular embodiment, the present invention relates to a combination as defined above, in which the oil is an oil powder as defined above.

According to another particular embodiment, the present invention relates to a combination as defined above, in which the oil is a fermented oil as defined above.

According to another particular embodiment, the present invention relates to a combination as defined above, in which the surfactant is chosen from: lauryl glucoside, decyl glucoside, caprylyl/capryl glucoside, coco-glucoside, hexyl glucoside, sodium cocoyl glutamate, disodium cocoyl glutamate, sodium stearoyl glutamate, sodium lauroyl glutamate, sodium myristoyl glutamate, coco-betaine, cocamidopropyl betaine, disodium cocoamphodiacetate, sodium arganamphoacetate, sodium babassuamphoacetate, sodium cocoabutteramphoacetate, sodium cottonseedamphoacetate, sodium sheabutteramphoacetate, sodium mangoamphoacetate, sodium olivamphoacetate, sodium ricebranamphoacetate, sodium sweetalmondamphoacetate, sodium sunflowerseedamphoacetate, potassium cocoate, potassium palmate, potassium laurate, potassium olivate, sodium coco-sulfate, sodium cocoamphoacetate, disodium coco-glucoside citrate, sodium coco-glucoside tartrate, sodium lauroamphoacetate, cocoyl methyl glucamide, capryloyl/caproyl methyl glucamide, lauroyl/myristoyl methyl glucamide, ammonium lauryl sulfate, ammonium coco-sulfate, magnesium lauryl sulfate, sodium C12-18 alkyl sulfate, zinc coco-sulfate, sodium cocoyl alaninate, sodium lauryl sulfate, sodium cetearyl sulfate, sodium cocoyl isethionate, sodium methyl oleoyl taurate, sodium methyl cocoyl taurate, cocamide MEA, cocamide MIPA, sodium C14-16 olefin sulfonate, sodium lauryl sulfoacetate, sodium lauroyl sarcosinate, sodium laureth sulfate, ammonium laureth sulfate, cocamidopropyl hydroxysultaine, disodium laureth sulfosuccinate, disodium lauryl sulfosuccinate, sodium myreth sulfate, laureth-5 carboxylic acid and saponin.

According to another particular embodiment, the present invention relates to a combination as defined above, in which the surfactant is an anionic surfactant, chosen in particular from: sodium cocoyl glutamate, and sodium cocoyl isethionate, or a mixture of these.

According to another particular embodiment, the present invention relates to a combination as defined above, in which the total quantity of powder adhesion agent is comprised from 5 to 85%, in particular from 5 to 15%, or from 70 to 85%, as a percentage by weight relative to the total weight of the combination.

With respect to the total quantity of adhesion agent, the term “from 5 to 85%” also means the following ranges: from 5 to 80%, from 5 to 70%, from 5 to 60%, from 5 to 50%, from 5 to 40%, from 5 to 30%, from 5 to 20%, from 5 to 15%, from 10 to 85%, from 20 to 85%, from 30 to 85%, from 40 to 85%, 50 to 85%, 50 to 85%, 60 to 85%, 70 to 85%, 80 to 85%, 10 to 80%, 20 to 70%, 30 to 60%, 40 at 50%.

According to another particular embodiment, the present invention relates to a combination as defined above, in which the total quantity of mannitol is comprised from 5 to 20%, in particular from 5 to 15% in percentage by weight relative to the weight total of the combination.

According to another particular embodiment, the present invention relates to a combination as defined above, in which the total quantity of α-glucan oligosaccharide is between 0.5 and 2%, in particular approximately 1% in percentage by weight relative to the total weight of the combination.

With respect to the total quantity of α-glucan oligosaccharide, the term “from 0.5 to 2%” also means the following ranges: from 0.6 to 2%, from 0.8 to 2%, from 1 to 2%, from 1.2 to 2%, from 1.4 to 2%, from 1.6 to 2%, from 1.8 to 2%, from 0.5 to 1.8%, from 0.5 to 1.6%, 0.5 to 1.4%, 0.5 to 1.2%, 0.5 to 1.0%, 0.5 to 0.8%, 0.6 to 1.8%, from 0.7 to 1.6%, from 0.8 to 1.4%, from 0.9 to 1.2%.

According to another particular embodiment, the present invention relates to a combination as defined above, in which the total quantity of oil is comprised from 1 to 50%, in particular from 1 to 10%, in particular from 2 to 8% in percentage by weight relative to the total weight of the combination.

With respect to the total quantity of oil, the term “from 1 to 50%” also means the following ranges: from 5 to 50%, from 10 to 50%, from 20 to 50%, from 30 to 50%, from 40 to 50%, 1 to 40%, 1 to 30%, 1 to 20%, 1 to 10%, 5 to 40%, 10 to 30%, 15 to 20%.

According to another particular embodiment, the present invention relates to a combination as defined above, in which the total quantity of surfactant is comprised from 1 to 80%, in particular from 2 to 20%, or from 40 to 80% by percentage by weight relative to the total weight of the combination.

With respect to the total amount of surfactant, the term “from 1 to 80%” also means the following ranges: from 10 to 80%, from 20 to 80%, from 30 to 80%, from 40 to 80%, from 50 to 80%, from 60 to 80%, from 70 to 80%, from 1 to 70%, from 1 to 60%, from 1 to 50%, from 1 to 40%, from 1 to 30%, from 1 to 20%, 1 to 10%, 5 to 10%, 10 to 70%, 20 to 60%, 30 to 50%.

According to another particular embodiment, the present invention relates to an combination as defined above, said combination being in the form:
of a shampoo, said shampoo comprising in particular:

two surfactants in a total amount of 40 to 80%, in particular 70 to 80%, as a percentage by weight relative to the total weight of the composition, and a powder adhesion agent, in an amount of 5 to 15% in percentage by weight relative to the total weight of the composition,

or
of a cleaner, said cleaner comprising in particular:

a surfactant in an amount of 2 to 20%, in particular 5 to 10% by weight percentage relative to the total weight of the composition, and a powder adhesion agent, in an amount of 70 to 85% in percentage by weight relative to the total weight of the composition.

The present invention also relates to a combination consisting of the following 4 constituents:
a powder adhesion agent,
α-glucan oligosaccharide,
at least one oil, and
at least one surfactant,

said combination being in powder form,

in which the mass percentage of water is between 0 and 10% relative to the total mass of the combination,

said adhesion agent, said α-glucan oligosaccharide, and said at least one surfactant being in powder form, said at least one oil being in particular in powder form.

According to this embodiment, the 4 constituents are as defined above.

Another object of the present invention is the use of a combination as defined above, in a cosmetic composition.

Another object of the present invention is a cosmetic composition, comprising or consisting of the combination as defined above, in a cosmetically acceptable medium,

said composition being in the form of a powder substantially free of water.

By “a cosmetically acceptable medium”, is meant within the meaning of the invention a medium compatible with a use in cosmetics.

According to a particular embodiment, the present invention relates to a cosmetic composition as defined above, comprising a combination as defined above, in a cosmetically acceptable medium,

said composition being in the form of a powder,

in which the mass percentage of water is between 0 and 10% relative to the total mass of the composition.

According to a particular embodiment, the present invention relates to a cosmetic composition, comprising a combination as defined above, in a cosmetically acceptable medium,

said composition being in the form of a powder,

in which the mass percentage of water is between 0 and 10% relative to the total mass of the composition,

wherein said composition further comprises a pigment or piroctone olamine.

According to this embodiment, the combination included in the cosmetic composition is free of mannitol.

According to a particular embodiment, the present invention relates to a cosmetic composition as defined above, comprising the following 5 constituents:
a powder adhesion agent,
mannitol,
α-glucan oligosaccharide,
at least one oil, and
at least one surfactant,

in which the mass percentage of water relative to the total mass of the cosmetic composition is between 0 and 10%, in particular from 0.1 to 10%, in particular from 0.1 to 5%,

said cosmetic composition being in powder form,

said adhesion agent, said mannitol, said α-glucan oligosaccharide, and said at least one surfactant being in powder form, said at least one oil being in particular in powder form,

wherein said cosmetic composition further comprises at least one ingredient,

the total weight of said at least one ingredient is in particular comprised from 0 to 15% by mass relative to the total weight of the composition, in particular from 0 to 10%, or from 2 to 15%.

According to another particular embodiment, the present invention relates to a cosmetic composition as defined above, in which the density of the powder is between 0.35 and 0.55 g/ml, in particular from 0.4 to 0.5 g/ml.

According to another particular embodiment, the present invention relates to a cosmetic composition as defined above, said combination being present in a proportion of 70 to 100%, in particular of 85 to 100% in percentage by weight relative to the total weight of the composition.

According to another particular embodiment, the present invention relates to a cosmetic composition as defined above, in which the total quantity of powder adhesion agent is comprised from 3.5 to 85%, in particular from 3.5 to 15%, or from 50 to 85%, in percentage by weight relative to the total weight of the composition.

With respect to the total quantity of adhesion agent of the powders, the term “from 3.5 to 85%” also means the following ranges: from 3.5 to 80%, from 3.5 to 70%, from 3.5 to 60%, 3.5 to 50%, 3.5 to 40%, 3.5 to 30%, 3.5 to 20%, 3.5 to 15%, 10 to 85%, 20 to 85%, 30 to 85%, 40 to 85%, 50 to 85%, 50 to 85%, 60 to 85%, 70 to 85%, 80 to 85%, from 10 to 80%, from 20 to 70%, from 30 to 60%, from 40 to 50%.

According to another particular embodiment, the present invention relates to a cosmetic composition as defined above, in which the total quantity of mannitol is comprised from 3.5 to 20%, in particular from 8 to 15%, in percentage by weight relative to the total weight of the composition.

According to another particular embodiment, the present invention relates to a cosmetic composition as defined above, in which the total quantity of α-glucan oligosaccharide is comprised from 0.35 to 2%, in particular approximately 1%, as a percentage by weight relative to the total weight of the composition.

With respect to the total quantity of α-glucan oligosaccharide, the term “from 0.35 to 2%” also means the following ranges: from 0.4 to 2%, from 0.6 to 2%, from 0.8 to 2%, from 1 to 2%, from 1.2 to 2%, from 1.4 to 2%, from 1.6 to 2%, from 1.8 to 2%, from 0.35 to 1.8%, 0.35 to 1.6%, 0.35 to 1.4%, 0.35 to 1.2%, 0.35 to 1.0%, 0.35 to 0.8%, from 0.35 to 0.5%, from 0.4 to 1.8%, from 0.6 to 1.6%, from 0.8 to 1.4%, from 1.0 to 1.2%.

According to another particular embodiment, the present invention relates to a cosmetic composition as defined above, in which the total quantity of oil is comprised from 0.7 to 50% in percentage by weight relative to the total weight of the composition.

With respect to the total quantity of oil, the term “from 0.7 to 50%” also means the following ranges: from 1 to 50%, from 5 to 50%, from 10 to 50%, from 20 to 50%, from 30 to 50%, from 40 to 50%, from 0.7 to 40%, from 0.7 to 30%, from 0.7 to 20%, from 0.7 to 10%, from 0.7 to 5%, 5 to 40%, 10 to 30%, 15 to 20%.

According to another particular embodiment, the present invention relates to a cosmetic composition as defined above, in which the total amount of surfactant is comprised from 0.7 to 80%, in particular from 1.4 to 20%, or comprised from 30 to 80% in percentage by weight relative to the total weight of the composition.

With respect to the total amount of surfactant, the term “from 0.7 to 80%” also means the following ranges: from 10 to 80%, from 20 to 80%, from 30 to 80%, from 40 to 80%, 50 to 80%, 60 to 80%, 70 to 80%, 1 to 70%, 1 to 60%, 1 to 50%, 1 to 40%, 1 to 30%, 1 at 20%, from 1 to 10%, from 10 to 70%, from 20 to 60%, from 30 to 50%.

According to another particular embodiment, the present invention relates to a cosmetic composition as defined above, said composition being free of: preservatives, such as phenylethanol, of parabens, of polyethylene glycol (PEG), of sulphates, of talc, of silicones, of allergens, of synthetic fragrances, and of irritating active ingredients.

The cosmetic composition of the present invention is in particular free of mineral salts of heavy metals.

Said preservatives, mineral salts of heavy metals, parabens, polyethylene glycol (PEG), sulphates, talc, silicones, allergens, synthetic fragrances, and irritating active agents are as defined above.

The cosmetic compositions according to the present invention are in particular free of non-natural materials or of non-natural origin, and of animal materials.

The cosmetic compositions according to the present invention may also comprise at least one ingredient as defined above.

According to a particular embodiment, the present invention relates to a cosmetic composition as defined above, in which

the composition also comprises at least one ingredient, in particular an active principle, in particular chosen from: flower acid, Zinc PCA, a pigment, the coal, an antioxidant, in particular vitamin C, or one of its derivatives, powdered tea, including matcha green tea, fermented tea, powdered kombucha tea, centella asiatica, or madecassoside, sugars, in particular xylitol, rhamnose, or fructooligosaccharides, the aloe vera, a protein, in particular pea protein, soy protein, wheat protein, or rice protein, sweet almond milk, or one or more of its components, in particular prunus dulcis, rice syrup or acacia fibre, a perfume, in particular an apricot almond perfume, ghassoul, white clay, especially kaolin sodium hyaluronate, hyaluronic acid, lactic acid, vanilla macerate, maltodextrin, bamboo powder, spirulina, phycocyanin, fermented mushrooms, guarana, caffeine, acerola, chamomile, Ayurvedic powders, vitamins, in particular vitamin B3, or vitamin B5, glycosphingolipids and glycolipids, for example from the Ceramosides™ HP brand (INCI: Glycosphingolipids, glycolipids), exopolysaccharides, and postbiotics, in particular lactobacillus ferment, such as the Lactobiotyl® brand (INCI: Maltodextrin and Lactobacillus Ferment), pyroctolamine, piroctone olamine, a camellia seed extract (INCI: Camellia oleifera seed extract), a corn-flower extract (INCI: Centaurea cyanus flower extract), a chamomile flower extract (INCI Maltodextrin, Chamomilla Recutita Flower Extract), or a mixture of these,

said at least one ingredient is in particular solid at room temperature.

According to another particular embodiment, the present invention relates to a cosmetic composition as defined above, said composition being in the form:
a shampoo, said shampoo comprising in particular:

two surfactants in a total amount comprised from 30 to 80%, in particular from 50 to 80% as a percentage by weight relative to the total weight of the composition, and a powder adhesion agent, in an amount comprised from 3.5 to 15% in percentage by weight relative to the total weight of the composition,

or
a cleaner, said cleaner comprising in particular:

a surfactant at a rate of 1.4 to 20% in percentage by weight relative to the total weight of the composition, and a powder adhesion agent, in an amount comprised from 50 to 85%, in particular from 70 to 85% in percentage by weight relative to the total weight of the composition.

According to a preferred embodiment, the present invention relates to a cosmetic composition as defined above, in which said composition comprises, in percentage by weight relative to the total weight of the composition:
a powder adhesion agent, in particular corn starch, at a rate of 8 to 11%, and
mannitol at a rate of 9 to 11%,
α-glucan oligosaccharide, at a rate of 0.5 to 1.5%,
Argan oil, at a rate of 3 to 5%,
sodium cocoyl glutamate at a rate of 25 to 35%,
sodium cocoyl isethionate at a rate of 35 to 45%,
aloe vera at a rate of 0.5 to 1%,
pea protein at 2 to 3%, and
apricot almond flavor at a rate of 1 to 3%,

said composition being in particular a shampoo.

According to another preferred embodiment, the present invention relates to a cosmetic composition as defined above, in which said composition comprises, in percentage by weight relative to the total weight of the composition:
a powder adhesion agent, in particular corn starch, at a rate of 10 to 12%,
mannitol at 9 to 10%,
α-glucan oligosaccharide, at a rate of 0.5 to 1.5%,
sweet almond milk, 6 to 7%,
sodium cocoyl glutamate at a rate of 25 to 35%, and
sodium cocoyl isethionate at a rate of 35 to 45%,

said composition being in particular a shampoo.

In this particular embodiment, the at least one oil is included in sweet almond milk which includes sweet almond oil.

According to another preferred embodiment, the present invention relates to a cosmetic composition as defined above, in which said composition comprises, in percentage by weight relative to the total weight of the composition:
a powder adhesion agent, in particular corn starch, at a rate of 8 to 11%,
mannitol at a rate of 7 to 9%,
α-glucan oligosaccharide, at a rate of 0.5 to 1.5%,
Ghassoul, at a rate of 4 to 6%,
sodium cocoyl glutamate at a rate of 25 to 35%,
sodium cocoyl isethionate at a rate of 35 to 45%,
bamboo powder at a rate of 2 to 4%,
jojoba oil at a rate of 1 to 3%, and
herbal and tea perfume at a rate of 1 to 3%,

said composition being in particular a shampoo.

According to another preferred embodiment, the present invention relates to a cosmetic composition as defined above, in which said composition comprises, in percentage by weight relative to the total weight of the composition:
sodium cocoyl glutamate at a rate of 25 to 35%,
sodium cocoyl isethionate at a rate of 35 to 45%,
a powder adhesion agent, in particular corn starch,
α-glucan oligosaccharide, at a rate of 0.5 to 1.5%,
sunflower oil,
chamomile flower extract,
piroctone olamine
flower acid, and
herbal and tea fragrance,

said composition being in particular a shampoo.

According to another preferred embodiment, the present invention relates to a cosmetic composition as defined above, in which said composition comprises, in percentage by weight relative to the total weight of the composition:
sodium cocoyl glutamate at a rate of 25 to 35%,
sodium cocoyl isethionate at a rate of 35 to 45%,
a powder adhesion agent, in particular corn starch,
α-glucan oligosaccharide, at a rate of 0.5 to 1.5%,
argan oil,
rice protein,
pea protein,
benzene sulfonic acid 2-[(9,10-dihydro-4-hydroxy-9,10-dioxo-1-anthracenyl) amino]-5-methyl monosodium salt,
erythrol, and
herbal and tea fragrance,

said composition being in particular a shampoo.

According to another preferred embodiment, the present invention relates to a cosmetic composition as defined above, in which said composition comprises, in percentage by weight relative to the total weight of the composition:
a powder adhesion agent, in particular corn starch, at a rate of 75 to 79%,
mannitol at a rate of 12 to 14%,
α-glucan oligosaccharide, at a rate of 0.5 to 1.5%,
sodium cocoyl glutamate at a rate of 4 to 6%,
sesame oil at a rate of 0.5 to 2%,
jojoba oil at a rate of 0.5 to 2%,
zinc PCA at a rate of 0.5 to 2%, and
coal at a rate of 0.5 to 2%,

said composition being in particular a cleanser.

According to another preferred embodiment, the present invention relates to a cosmetic composition as defined above, in which said composition comprises, in percentage by weight relative to the total weight of the composition:
a powder adhesion agent, in particular corn starch, at a rate of 72 to 76%,
mannitol at a rate of 9 to 11%,
α-glucan oligosaccharide, at a rate of 0.5 to 1.5%,
sodium cocoyl glutamate at a rate of 4 to 6%,
sweet almond oil at a rate of 2 to 4%,
ascorbyl glucoside at a rate of 2 to 4%,
jojoba oil at 2 to 4%, and
flower acid at a rate of 1 to 3%,

said composition being in particular a cleanser.

According to another preferred embodiment, the present invention relates to a cosmetic composition as defined above, in which said composition comprises, in percentage by weight relative to the total weight of the composition:
a powder adhesion agent, in particular corn starch, at a rate of 72 to 76%,
mannitol at 11 to 13%,
α-glucan oligosaccharide, at a rate of 0.5 to 1.5%,
sodium cocoyl glutamate at a rate of 4 to 6%,
camellia oil at a rate of 2 to 4%,
jojoba oil at a rate of 1 to 3%, and
matcha tea at a rate of 1 to 3%,

said composition being in particular a cleanser.

According to another preferred embodiment, the present invention relates to a cosmetic composition as defined above, in which said composition comprises, in percentage by weight relative to the total weight of the composition:
a powder adhesion agent, in particular corn starch, at a rate of 65 to 75%,
mannitol at a rate of 14 to 16%,
α-glucan oligosaccharide, at a rate of 0.5 to 1.5%,
sodium cocoyl glutamate at a rate of 4 to 6%,
safflower oil at a rate of 2 to 4%,
jojoba oil at 2 to 4%, and
spirulina at a rate of 2 to 4%,

said composition being in particular a cleanser.

According to another preferred embodiment, the present invention relates to a cosmetic composition as defined above, in which said composition comprises, in percentage by weight relative to the total weight of the composition:
a powder adhesion agent, in particular corn starch, at a rate of 70 to 76%,
mannitol at a rate of 13 to 17%,
α-glucan oligosaccharide, at a rate of 0.5 to 1.5%,
sodium cocoyl glutamate at a rate of 4 to 6%,
safflower oil at a rate of 1 to 3%,
argan oil at a rate of 1 to 3%,
hemp oil at a rate of 1 to 3%, and

said composition being in particular a cleanser.

According to another particular embodiment, the present invention relates to a combination as defined above, or a cosmetic composition as defined above, said combination or composition being formulated in a capsule.

The capsule that can be used in the present invention is in particular a water-soluble capsule, said capsule being able to be dissolved in water during use, thus releasing the contents of the capsule. Alternatively, if the capsule is not water soluble, the contents can be released by opening the capsule. The person skilled in the art will be able to choose a capsule according to the need from among the commercially available capsules, in particular in the pharmaceutical field, or food supplements.

Like the constituents and ingredients of the combination and those of the cosmetic composition of the present invention, the capsules are made from natural materials and of non-animal origin, such as for example hydroxymethyl cellulose, alginates or starch. The size of the capsules can be chosen according to the total quantity of product to be contained.

According to another particular embodiment, the present invention relates to a combination or a cosmetic composition as defined above, in which said combination or said composition is included in a capsule, in an amount of 0.5 to 2.5 g, preferably from 0.75 to 1.25 g, or from 1.75 to 2.25 g, in particular approximately 1 g, or 2 g.

When applying the combination or the cosmetic composition, an ephemeral aqueous solution is obtained, in which the constituents and the ingredients are in solution. Certain ingredients, such as coal or ghassoul, for example, may however be suspended in said ephemeral solution.

Another object of the present invention is an aqueous solution comprising a combination as defined above, or a cosmetic composition as defined above, said combination or composition being present in a concentration of approximately 10 g/L, said aqueous solution having a pH of 4 to 6.

By “approximately 10 g/L” is meant a value comprised from 9 to 11 g/L.

An aqueous solution comprising a cosmetic combination or composition has a pH compatible with cosmetic use on the skin in the sense that the pH of the solution is close to the pH of the skin.

Another object of the present invention is a device comprising
a capsule, in particular a water-soluble capsule, and
a combination as defined above, or a cosmetic composition as defined above, in particular in an amount of 0.5 to 2.5 g, preferably 0.75 to 1.25 g, or 1.75 to 2.25 g, in particular about 1 g, or 2 g.

said combination or cosmetic composition being contained within the capsule.

The cleansers according to the present invention are used in an amount comprised of 1 to 2 grams, in particular approximately 1 gram, while the shampoos are used in an amount of approximately 1 gram for short hair and approximately 2 grams for long hair.

Another object of the present invention is the use of a combination as defined above, or of a cosmetic composition as defined above, or of a device as defined above, for the hair or skin care.

Another object of the present invention is a cosmetic treatment method comprising:
a step of solubilizing a combination as defined above, or a cosmetic composition as defined above, or a device as defined above, by adding water, to obtain an aqueous solution, and
a step of applying said aqueous solution to the hair or to the skin,

the total amount of water used in the solubilization step is in particular between 300 and 500% in percentage by weight relative to the weight of the combination, of the cosmetic composition, or of the device.

Another object of the present invention is a cosmetic treatment method comprising:
a step of opening the capsule, in the palm of the hand, to release the combination as defined above, or the cosmetic composition as defined above,
a step of dissolving said combination, or said cosmetic composition, by adding water, to obtain an aqueous solution, and
a step of applying said aqueous solution to the hair or to the skin,

the total quantity of water used in the solubilization step being in particular comprised from 300 to 500% in percentage by weight relative to the weight of the combination or of the cosmetic composition.

The inventors have found that the shampoos of the present invention, comprising in particular two surfactants, have advantageous properties, in particular with respect to sensory characteristics such as foaming power, washing power, detangling power, rinsability and ease of combing.

The inventors have also found that the cleansers of the present invention, comprising in particular a single surfactant, have advantageous properties, in particular with respect to sensory characteristics such as foaming power in contact with water, make-up removing power, and rinsability.

The following examples illustrate the invention, without limiting its scope.

### EXAMPLES

### Example a General Procedure for the Manufacture of the Compositions

The different ingredients are weighed and added one by one in a mixer. The powders are added first and the vegetable oil and perfume are added second. The ingredients are mixed for 10 seconds twice and then packaged.

### Example B Powder Density Measurement Procedure

A 250 ml test tube was weighed empty to give its weight M0 in grams. A volume V of the powder, ranging from 240 to 250 ml, is versed into the test tube using a funnel. The test tube is weighed again to give the weight M1 in grams.

The density ρ of the powder, expressed in g/ml, is calculated according to the following formula 1:

ρ=(M1−M0)/V formula 1

### Example C pH Measurement Procedure

The composition is weighed and diluted in water to obtain an aqueous solution of the composition at 10% by weight. The pH is then measured at room temperature, using a pH meter.

### Example D General Procedure for Cleaning Sensory Tests on a Panel of Experts

Sensory tests were carried out on a panel of experts.

The powder cleanser was used like a regular facial cleanser. It was used as follows:
1) Wet the face
2) Sprinkle 1 teaspoon of the powder cleanser into your hand
3) Add a few drops of water to make it foam in your hands
4) Apply cleanser to face and massage to remove makeup and impurities
5) Rinse with water

The performance of the cleaner is evaluated according to:
its foaming power in contact with water
its make-up removing power
its rinsability

### Example E General Procedure for Shampoo Tests on a Panel of Experts

Sensory tests were carried out on a panel of experts.

The powder shampoo was used on wet hair like a classic shampoo. It was used as follows:
1) Wet her hair and hands
2) Sprinkle 1 to 2 teaspoons of shampoo powder into hand depending on length of hair and add water if needed
3) Apply shampoo to hair
4) Massage hair and scalp
5) Rinse with water

The performance of the shampoo is evaluated according to:
its foaming power
its cleansing power
its detangling power
its rinsability
its ease of combing

### Example 1 Ghassoul Shampoo

The shampoo composition of Table 1, comprising Ghassoul, was manufactured according to the general procedure of Example A.

TABLE 1
Composition of a Ghassoul shampoo

| name | commercial name | INCI name | % |
|---|---|---|---|
| Corn strach | MAISITA 9060 | Zea Mays (Corn) Starch | 9 |
| Mannitol | Beauté by Roquette ® PO260 | Mannitol | 8 |
| Sodium cocoyl | AMISOFT ® CS 11 | SODIUM COCOYL | 30 |
| glutamate | | GLUTAMATE | |
| Sodium cocoyl | PUREACT I-80P | SODIUM COCOYL | 40 |
| iséthionate | | ISETHIONATE | |
| Ghassoul | GHASSOUL | Morrocan Lava Clay | 5 |
| Bamboo powder | Greensil | Bambusa Arundinacea Stem | 3 |
| | | Extract | |
| alpha-Glucan | BIOECOLIA ® | α-glucan oligosaccharide | 1 |
| oligosaccharide | | | |
| Jojoba oil | Jojoba Oil CP Org | SIMMONDSIA CHINENSIS | 2 |
| | | SEED OIL | |
| Herb & Tea fragrance | HERBES & THE 2 NATFLOR | | 2 |
| | RT4693 | | |

| | |
|---|---|
| TOTAL | 100 |

The composition is in the form of a white powder, the density of which, measured according to the procedure of Example B, is 0.42 g/ml.

The pH, measured according to the procedure of Example C, is 5.85.

Sensory Tests

The shampoo was evaluated according to the procedure of Example E:
foaming power: Foam formation is rapid. The foam is abundant, light and pleasant to the touch.
washing power: The hair is clean after washing.
detangling power: The hair is easy to detangle.
rinsability: The shampoo rinses out very quickly.
ease of combing: The hair is easy to detangle when combing.

### Example 2 Ghassoul Shampoo

The shampoo composition of Table 2, comprising Ghassoul, was manufactured according to the general procedure of Example A.

TABLE 2
Composition of a Ghassoul shampoo

| name | INCI name | % |
|---|---|---|
| Corn starch | Zea mays (corn) starch | 8 |
| Mannitol | Mannitol | 8 |
| Sodium cocoyl glutamate | Sodium cocoyl glutamate | 30 |
| Sodium cocoyl iséthionate | Sodium cocoyl isethionate | 40 |
| Ghassoul | Maroccan lava clay | 5 |
| L-Arginin | L-Arginine | 1 |
| Bbamboo powder | Bambusa arundinacea stem | 3 |
| | powder | |
| Jojoba oil | Simmondsia chinensis seed oil | 2 |
| alpha-Glucan oligosaccharide | α-glucan oligosaccharide | 1 |
| Herb & tea fragrance | fragrance | 2 |
| | Total | 100 |

The composition is in the form of a white powder, the density of which, measured according to the procedure of Example B, is 0.43 g/ml.

The pH, measured according to the procedure of Example C, is 5.7.

Sensory Tests

The shampoo was evaluated according to the procedure of Example E:
foaming power: Foam formation is rapid. The foam is abundant, light and pleasant to the touch.
washing power: The hair is clean after washing.
detangling power: The hair is easy to detangle.
rinsability: The shampoo rinses out very quickly.
ease of combing: The hair is easy to detangle when combing.

### Example 3 Argan Oil Shampoo

The shampoo composition of Table 3, comprising argan oil, was manufactured according to the general procedure of Example A.

TABLE 3
Composition of an argan oil shampoo

| Name | commercial name | INCI name | % |
|---|---|---|---|
| Corn starch | MAISITA 9060 | Zea Mays (Corn) Starch | 9.5 |
| Mannitol | Beauté by Roquette ® PO260 | Mannitol | 10 |
| Sodium cocoyl | AMISOFT ® CS 11 | SODIUM COCOYL | 30 |
| glutamate | | GLUTAMATE | |
| Sodium cocoyl | PUREACT I-80P | SODIUM COCOYL | 40 |
| isethionate | | ISETHIONATE | |
| Pea protein | Promois WJ-SP | Hydrolyzed Pea Protein | 2.5 |
| Aloe vera | ALOE VERA EXTRAIT SEC | Aloe Barbadensis Leaf Juice | 1 |
| | ATOMISE BIO | Powder | |
| α-glucan | BIOECOLIA ® | α-glucan oligosaccharide | 1 |
| oligosaccharide | | | |
| Argan oil | Deodorized argan oil | Argania Spinosa Kernel Oil | 4 |
| Almond apricot | ABRICOT AMANDE 2 | | 2 |
| fragrance | NATFLOR RT4688 | | |

| | |
|---|---|
| TOTAL | 100 |

The composition is in the form of a white powder, the density of which, measured according to the procedure of Example B, is 0.40 g/ml.

The pH, measured according to the procedure of Example C, is 5.49.

### Example 4 Argan Oil Shampoo

The shampoo composition of Table 4, comprising argan oil, was manufactured according to the general procedure of Example A.

TABLE 4
Composition of an argan oil shampoo

| Name | INCI name | % |
|---|---|---|
| Corn starch | Zea Mays (Corn) Starch | 10 |
| Mannitol | Mannitol | 10 |
| Sodium cocoyl | SODIUM COCOYL GLUTAMATE | 30 |
| glutamate | | |
| Sodium cocoyl | SODIUM COCOYL ISETHIONATE | 40 |
| isethionate | | |
| Pea protein | Hydrolyzed Pea Protein | 2 |
| Aloe vera | Aloe Barbadensis Leaf Juice Powder | 1 |
| α-glucan | α-glucan oligosaccharide | 1 |
| oligosaccharide | | |
| Argan oil | Argania Spinosa Kernel Oil | 4 |
| fragrance | | 2 |

| | |
|---|---|
| TOTAL | 100 |

The composition is in the form of a white powder, the density of which, measured according to the procedure of Example B, is 0.34 g/ml.

The pH, measured according to the procedure of Example C, is 5.8.

Sensory Tests

The shampoo was evaluated according to the procedure of Example E:
foaming power: Foam formation is rapid. The foam is abundant, light and pleasant to the touch.
washing power: The hair is clean after washing.
detangling power: The hair is easy to detangle.
rinsability: The shampoo rinses out very quickly.
ease of combing: The hair is easy to detangle when combing.

### Example 5 Sweet Almond Milk Shampoo

The shampoo composition of Table 5, comprising sweet almond milk, was manufactured according to the general procedure of Example A.

TABLE 5
Composition of a sweet almond milk shampoo

| name | Commercial name | INCI name | % |
|---|---|---|---|
| Corn starch | MAISITA 9060 | Zea Mays (Corn) Starch | 11 |
| Mannitol | Beauté by Roquette PO 260 | Mannitol | 9.5 |
| Sodium cocoyl | AMISOFT ® CS 11 | SODIUM COCOYL | 30 |
| glutamate | | GLUTAMATE | |
| Sodium cocoyl | PUREACT I-80P | SODIUM COCOYL | 40 |
| isethionate | | ISETHIONATE | |
| Sweet almond milk | LAIT AMANDE DOUCE BIO | Prunus Amygdalus Dulcis | 6.5 |
| | | Protein | |
| | | Oryza Sativa Extract | |
| | | Acacia Senegal Gum | |
| | | Vanilla Planifolia Extract | |
| | | Prunus Amygdalus Dulcis Fruit | |
| | | Extract | |
| α-glucan | BIOECOLIA ® | α-glucan oligosaccharide | 1 |
| oligosaccharide | | | |
| Coco milk fragrance | LAIT DE COCO 2 NATFLOR | | 2 |
| | RT4683 | | |

| | |
|---|---|
| TOTAL | 100 |

The composition is in the form of a white powder, the density of which, measured according to the procedure of Example B, is 0.48 g/ml.

The pH, measured according to the procedure of Example C, is 5.84.

### Example 6 Sweet Almond Milk Shampoo

The shampoo composition of Table 6, comprising sweet almond milk, was manufactured according to the general procedure of Example A.

TABLE 6
Composition of a sweet almond milk shampoo

| name | INCI name | % |
|---|---|---|
| Corn starch | Zea mays (corn) starch | 11 |
| Mannitol | Mannitol | 9.5 |
| Sodium cocoyl glutamate | Sodium cocoyl glutamate | 30 |
| Sodium cocoyl isethionate | Sodium cocoyl isethionate | 40 |
| Sweet almond milk | Lait d'amande douce | 4 |
| Hydrolized rice protein | Hydrolyzed rice bran protein | 2 |
| Kappaphycus alvarezii | Kappaphycus alvarezii Extract & | 0.5 |
| extract & tara fruit extract | Caesalpinia spinosa Fruit Extract | |
| α-glucan oligosaccharide | α-glucan oligosaccharide | 1 |
| fragrance | | 2 |
| | Total | 100 |

The composition is in the form of a white powder, the density of which, measured according to the procedure of Example B, is 0.44 g/ml.

The pH, measured according to the procedure of Example C, is 5.7.

Sensory Tests

The shampoo was evaluated according to the procedure of Example E:
foaming power: Foam formation is rapid. The foam is abundant, light and pleasant to the touch.
washing power: The hair is clean after washing.
detangling power: The hair is easy to detangle.
rinsability: The shampoo rinses out very quickly.
ease of combing: The hair is easy to detangle when combing.

### Example 7 Shampoo with 3 Oils

The shampoo composition of Table 7, comprising three oils, was made according to the general procedure of Example A.

TABLE 7
Composition of a shampoo with three oils

| name | Commercial name | INCI name | % |
|---|---|---|---|
| Corn starch | MAISITA 9060 | Zea Mays (Corn) Starch | 6 |
| Mannitol | Beauté by Roquette ® PO260 | Mannitol | 10 |
| Sodium cocoyl | AMISOFT ® CS 11 | SODIUM COCOYL | 30 |
| glutamate | | GLUTAMATE | |
| Sodium cocoyl | PUREACT I-80P | SODIUM COCOYL | 40 |
| iséthionate | | ISETHIONATE | |
| Aloe vera | ALOE VERA EXTRAIT SEC | Aloe Barbadensis Leaf Juice | 5 |
| | ATOMISE BIO | Powder | |
| α-glucan | BIOECOLIA ® | α-glucan oligosaccharide | 1 |
| oligosaccharide | | | |
| Moringa seed oil | ORGANIC REFINED | Moringa Oleifera Seed Oil | 2 |
| | MORINGA OIL | | |
| Jojoba oil | Jojoba Oil CP Org | SIMMONDSIA CHINENSIS | 2 |
| | | SEED OIL | |
| Argan oil | Huile d'argan désodorisée | Argania Spinosa Kernel Oil | 2 |
| | ABRICOT AMANDE 2 | | 2 |
| | NATFLOR RT4688 | | |

| | |
|---|---|
| TOTAL | 100 |

The composition is in the form of a white powder.

The pH, measured according to the procedure of Example C, is 5.71.

### Example 8 Charcoal Cleaner

The cleaning composition of Table 8, including charcoal, was made according to the general procedure of Example A.

TABLE 8
Composition of a charcoal cleaner

| Name | Commercial name | INCI name | % |
|---|---|---|---|
| Corn starch | MAISITA 9060 | Zea Mays (Corn) Starch | 77 |
| Mannitol | Beauté by Roquette ® PO260 | Mannitol | 13 |
| Jojoba oil | Jojoba Oil CP Org | SIMMONDSIA CHINENSIS | 1 |
| | | SEED OIL | |
| Sesame oil | HUILE VIERGE DE | Sesamum Indicum Seed Oil | 1 |
| | SESAME BIO | | |
| Coal powder | CHARBON VEGETAL | Charcoal Powder | 1 |
| | POUDRE | | |
| Zinc salt of | ZINCIDONE ® | Zinc PCA | 1 |
| pyrrolidone | | | |
| carboxylic acid | | | |
| α-glucan | BIOECOLIA ® | α-glucan oligosaccharide | 1 |
| oligosaccharide | | | |
| Sodium cocoyl | AMISOFT ® CS 11 | Sodium Cocoyl Glutamate | 5 |
| glutamate | | | |

| | |
|---|---|
| TOTAL | 100 |

The composition is in the form of a white powder, the density of which, measured according to the procedure of Example B, is 0.4 g/ml.

The pH, measured according to the procedure of Example C, is 5.3.

### Example 9 Charcoal Cleaner

The cleaning composition in Table 9, including coal, was made according to the general procedure of Example A.

| name | INCI name | % |
|---|---|---|
| arrowroot powder | Maranta Arundinacea Root Powder | 77 |
| Mannitol | Mannitol | 13 |
| Jojoba oil | SIMMONDSIA CHINENSIS SEED OIL | 1 |
| Sesame oil | Sesamum Indicum Seed Oil | 1 |
| Coal powder | coal Powder | 1 |
| Zinc salt of | Zinc PCA | 1 |
| pyrrolidone | | |
| carboxylic acid | | |
| α-glucan | α-glucan oligosaccharide | 1 |
| oligosaccharide | | |
| Sodium cocoyl | Sodium Cocoyl Glutamate | 5 |
| glutamate | | |

| | |
|---|---|
| TOTAL | 100 |

The composition is in the form of a white powder, the density of which, measured according to the procedure of Example B, is 0.39 g/ml.

The pH, measured according to the procedure of Example C, is 5.7.

Sensory Tests

The cleaner was evaluated according to the procedure of Example D:
foaming power: In contact with water, the cleanser foams slightly and becomes creamy.
make-up removing power: The cleanser cleanses the skin and removes make-up. However, it needs to be combined with an eye make-up remover to perfect eye make-up removal when the eyes are made up.
rinsability: The cleanser rinses off easily and leaves a feeling of softness on the skin after use.

### Example 10 Flower Acid Cleanser

The cleaning composition in Table 10, including coal, was made according to the general procedure of Example A.

TABLE 10
Composition of a flower acid cleanser

| name | Commercial name | INCI name | % |
|---|---|---|---|
| Corn starch | MAISITA 9060 | Zea Mays (Corn) Starch | 74 |
| Mannitol | Beauté by Roquette ® PO260 | Mannitol | 10 |
| Jojoba oil | Jojoba Oil CP Org | SIMMONDSIA CHINENSIS | 3 |
| | | SEED OIL | |
| Sweet almond oil | HUILE D'AMANDE DOUCE | Prunus Amygdalus Dulcis | 3 |
| | VIERGE BIO | (Sweet Almond) Oil | |
| Ascorbyl Glucoside | AA2G | Ascorbyl Glucoside | 2 |
| Flower acid | ACIDE DE FLEURS ROUGES | Hibiscus Sabdariffa Flower | 2 |
| | EXTRAIT SEC | Extract, Maltodextrin | |
| α-glucan | BIOECOLIA ® | α-glucan oligosaccharide | 1 |
| oligosaccharide | | | |
| Sodium cocoyl | AMISOFT ® CS 11 | Sodium Cocoyl Glutamate | 5 |
| glutamate | | | |

| | |
|---|---|
| TOTAL | 100 |

The composition is in the form of a white powder, the density of which, measured according to the procedure of Example B, is 0.41 g/ml.

The pH, measured according to the procedure of Example C, is 4.82.

### Example 11 Flower Acid Cleanser

The cleaning composition in Table 11, including coal, was made according to the general procedure of Example A.

TABLE 11
Composition of a flower acid cleanser

| name | INCI name | % |
|---|---|---|
| Arrowroot Powder | Maranta Arundinacea Root | 74 |
| | Powder | |
| Mannitol | Mannitol | 10 |
| Jojoba oil | SIMMONDSIA CHINENSIS SEED | 3 |
| | OIL | |
| Sweet almond oil | Prunus Amygdalus Dulcis (Sweet | 3 |
| | Almond) Oil | |
| Ascorbyl Glucoside | Ascorbyl Glucoside | 2 |
| Flower acid | Hibiscus Sabdariffa Flower | 2 |
| | Extract, Maltodextrin | |
| α-glucan | α-glucan oligosaccharide | 1 |
| oligosaccharide | | |
| Sodium cocoyl | Sodium Cocoyl Glutamate | 5 |
| glutamate | | |

| | |
|---|---|
| TOTAL | 100 |

The composition is in the form of a white powder, the density of which, measured according to the procedure of Example B, is 0.40 g/ml.

The pH, measured according to the procedure of Example C, is 5.

Sensory Tests

The cleaner was evaluated according to the procedure of Example D:
foaming power: In contact with water, the cleanser foams slightly and becomes creamy.
make-up removing power: The cleanser cleanses the skin and removes make-up. However, it needs to be combined with an eye make-up remover to perfect eye make-up removal when the eyes are made up.
rinsability: The cleanser rinses off easily and leaves a feeling of softness on the skin after use.

### Example 12 Tri-Oil Cleanser

The cleaning composition in Table 12, including oils, was made according to the general procedure of Example A.

TABLE 12
Composition of a three-oil cleanser

| name | Commercial name | INCI name | |
|---|---|---|---|
| Corn starch | MAISITA 9060 | Zea Mays (Corn) Starch | 73 |
| Mannitol | Beauté by Roquette ® PO260 | Mannitol | 15 |
| Oil starch | Huile d'argan désodorisée | Argania Spinosa Kernel Oil | 2 |
| Virgin Safflower Oil | Huile Vierge de Carthame Bio | Carthamus Tinctorius Seed Oil | 2 |
| Hemp oil | Organic Hemp Oil | Cannabis Sativa Seed Oil | 2 |
| α-glucan | BIOECOLIA ® | α-glucan oligosaccharide | 1 |
| oligosaccharide | | | |
| Sodium cocoyl | AMISOFT ® CS 11 | Sodium Cocoyl Glutamate | 5 |
| glutamate | | | |

| | |
|---|---|
| TOTAL | 100 |

The composition is in the form of a white powder, the density of which, measured according to the procedure of Example B, is 0.5 g/ml.

The pH, measured according to the procedure of Example C, is 5.5.

### Example 13 Matcha Green Tea Cleanser

The cleaning composition in Table 13, including matcha green tea, was made according to the general procedure of Example A.

| name | INCI name | % |
|---|---|---|
| arrowroot powder | Maranta Arundinacea Root | 74 |
| | Powder | |
| Mannitol | Mannitol | 12 |
| Jojoba oil | SIMMONDSIA CHINENSIS | 3 |
| | SEED OIL | |
| camellia oil | Camellia Oleifera Seed Oil | 3 |
| Matcha green tea | Camellia Sinensis Leaf Extract | 2 |
| α-glucan | α-glucan oligosaccharide | 1 |
| oligosaccharide | | |
| Sodium cocoyl | Sodium Cocoyl Glutamate | 5 |
| glutamate | | |

| | |
|---|---|
| TOTAL | 100 |

The composition is in the form of a white powder, the density of which, measured according to the procedure of Example B, is 0.43 g/ml.

The pH, measured according to the procedure of Example C, is 5.4.

Sensory Tests

The cleaner was evaluated according to the procedure of Example D:
foaming power: In contact with water, the cleanser foams slightly and becomes creamy.
make-up removing power: The cleanser cleanses the skin and removes make-up. However, it needs to be combined with an eye make-up remover to perfect eye make-up removal when the eyes are made up.
rinsability: The cleanser rinses off easily and leaves a feeling of softness on the skin after use.

### Example 14 Spirulina Cleanser

The cleansing composition in Table 14, including spirulina, was made according to the general procedure of Example A.

TABLE 14
Composition of a spirulina cleanser

| name | Commercial name | INCI name | % |
|---|---|---|---|
| arrowroot powder | | Maranta Arundinacea | 70 |
| | | Root Powder | |
| Mannitol | | Mannitol | 15 |
| Jojoba oil | | SIMMONDSIA | 3 |
| | | CHINENSIS SEED | |
| | | OIL | |
| Safflower Oil | | Carthamus Tinctorius | 3 |
| | | Seed Oil | |
| Spirulina | | Spirulina Platensis | 3 |
| | | Extract | |
| α-glucan | BIOECOLIA ® | α-glucan | 1 |
| oligosaccharide | | oligosaccharide | |
| Sodium cocoyl | | Sodium Cocoyl | 5 |
| glutamate | | Glutamate | |

| | |
|---|---|
| TOTAL | 100 |

The composition is in the form of a white powder, the density of which, measured according to the procedure of Example B, is 0.4 g/ml.

The pH, measured according to the procedure of Example C, is 5.7.

Sensory Tests

The cleaner was evaluated according to the procedure of Example D:
foaming power: In contact with water, the cleanser foams slightly and becomes creamy.
make-up removing power: The cleanser cleanses the skin and removes make-up. However, it needs to be combined with an eye make-up remover to perfect eye make-up removal when the eyes are made up.
rinsability: The cleanser rinses off easily and leaves a feeling of softness on the skin after use.

### Example 15 Anti-Dandruff Shampoo

The shampoo composition of Table 15 was manufactured according to the general procedure of Example A.

TABLE 15
Composition of an anti-dandruff shampoo

| name | Commercial name | INCI name | % |
|---|---|---|---|
| Sodium cocoyl | Pureact I-80P | Sodium cocoyl | 40 |
| isethionate | | isethionate | |
| Sodium cocoyl | Amisoft CS 11 | Sodium Cocoyl | 30 |
| glutamate | | Glutamate | |
| Corn starch | Maisita 9060 | Zea Mays (Corn) | 17 |
| | | Starch | |
| Sunflower oil | HUILE DE | HELIANTHUS | 4 |
| | TOURNESOL | ANNUS SEED OIL | |
| | DESODO BIO | | |
| Chamomile | Chamomile | Maltodextrin, | 2 |
| flower extract | Herbasec ® | Chamomilla Recutita | |
| | | Flower Extract | |
| α-glucan | Bioecolia ® | α-glucan | 3 |
| oligosaccharide | | oligosaccharide | |
| piroctone olamine | Kopirox | Piroctone Olamine | 1 |
| Flower acid | ACIDES DE | HIBISCUS | 1 |
| | FLEURS | SABDARIFFA | |
| | ROUGES | FLOWER EXTRACT, | |
| | EXTRAIT | MALTODEXTRIN | |
| | SEC (001233) | | |
| herb & tea | | | 2 |
| fragrance | | | |

| | |
|---|---|
| TOTAL | 100 |

### Example 16 Shampoo for Blonde Hair

The shampoo composition of Table 16 was manufactured according to the general procedure of Example A.

TABLE 16
Composition of a shampoo for blond hair

| name | Commercial name | INCI name | % |
|---|---|---|---|
| Sodium cocoyl | | Sodium cocoyl | 40 |
| isethionate | | isethionate | |
| Sodium cocoyl | | Sodium cocoyl | 30 |
| glutamate | | glutamate | |
| Erythriol | Erylite ® | Erythriol | 8 |
| Corn starch | | Zea Mays (Corn) | 10.5 |
| | | Starch | |
| Argan oil | | | 5 |
| Pea protein | | | 1.5 |
| Rice protein | | | 1.5 |
| herb & tea | | | 2 |
| fragrance | | | |
| α-glucan | Bioecolia ® | α-glucan | 1 |
| oligosaccharide | | oligosaccharide | |
| | Unicert violet K7025-J | CI 60730 | 0.5 |

| | |
|---|---|
| Total | 100 |

### Example 17 Tests for Evaluating the Clinical Tolerance of Two Shampoos

The following shampoos have been tested in a test to assess the clinical and dermatological tolerance and acceptability of a cosmetic product after use over a period of 21 days under normal conditions of use.
Almond milk shampoo according to example 5, on 21 volunteers
Argan oil shampoo according to example 3, on 22 volunteers

The tests were carried out by IDEA Clinic, France.

Instructions Selected for the Study

Use the shampoo according to your usual routine:
Shake well before use;
Apply in the shower, thoroughly wet your hair and hands;
Pour ½ teaspoon of shampoo powder into your hand depending on the length of your hair;
Add water to your hands to lather;
Apply to hair and lather like a classic shampoo;
Rinse well with water.

The volunteers evaluated the product for its properties after use over a period of 21 days. The study was monocentric, open and comparative between D21 and D1.

Results—Discussion

No clinical signs of intolerance were observed for the two shampoos.

Almond Milk Shampoo
Items relating to the efficacy of the product (positive responses≥75%, threshold of statistical significance for 21 volunteers analysed)
   The product is gentle on the hair as well as the scalp (85.71%)
   The product is suitable for your hair type (76.19%)
   The product gently cleanses the hair (90.48%)
   The product protects hair and scalp (85.71%)
   The hair is light (76.19%)
   The hair is supple (85.71%)
   The hair is soft and silky (80.95%)
   The hair is hydrated (85.71%)
   The hair is nourished (85.71%)
   The foam is melting (85.71%)
   The product is easy to rinse off (90.48%)
   The product rinses out more easily than a conventional shampoo (80.95%)

Under the conditions of the study, the product exhibited:
excellent dermatological tolerance, according to the scale adopted, on oily scalp and on normal and fine hair
good cosmetic acceptability, with 76% favorable opinions.

Argan Oil Shampoo
Items relating to the efficacy of the product (positive responses≥75%, threshold of statistical significance for 22 volunteers analysed)
   The product is easy to rinse off (90.91%)
   The product rinses out more quickly than a classic shampoo (77.27%)
   The product is gentle on the hair and the scalp (81.82%)
   The hair is light (77.27%)
   My color is preserved (77.27%)
   The scalp itches less (87.50%)

Under the conditions of the study, the product exhibited:
excellent dermatological tolerance, according to the scale adopted, on dry and colored hair and on the scalp;
good cosmetic acceptability, with 64% favorable opinions.

### Example 18 Clinical Tolerance Evaluation Tests of Three Cleansers

The following cleansers have been tested in a test to assess clinical and dermatological tolerance and acceptability of a cosmetic product after use over a period of 21 days under normal conditions of use.
The coal cleaner according to example 8, on 23 volunteers
The hibiscus flower acid cleanser according to example 10, on 22 volunteers
The three-oil cleanser according to example 12, on 23 volunteers

The tests were carried out by IDEA Clinic, France.

Instructions Selected for the Study

Once a day, preferably in the evening:
shake the cleaner before use,
pour ½ teaspoon of product into the hand,
add water and lather between your hands,
apply to the face, massaging in circular movements to cleanse it well and rinse well with water.

The volunteers evaluated the product for its properties after use over a period of 21 days. The study was monocentric, open and comparative between D21 and D1.

Results—Discussion

No clinical signs of intolerance were observed for the two shampoos.

Coal Cleaner
Items relating to the efficacy of the product (positive responses≥75%, threshold of statistical significance for 23 volunteers analysed)
   The product perfectly cleanses the skin (95.65%)
   The product is pleasant to use (100%)
   The product is easy to apply (95.65%)
   The product is easy to rinse off (100%)
   The skin is soft (95.65%)
   The skin is clearer (95.65%)
   The product removes impurities (95.65%)
   The skin is clean and healthy (95.65%)
   The product improves the overall appearance and condition of the skin (95.65%)
   The product reduces the feeling of tightness and provides a feeling of comfort (95.65%)
   The skin is purified (95.65%)
   Pores appear tightened (95.65%)
   The product reduces imperfections (86.96%)
   The product is gentle on the skin (95.65%)
   The skin is less shiny (95.65%)
   The product limits excess of sebum (95.65%)
   The product matifies the skin (95.65%)
   The skin is protected from external aggressions (95.65%)
   The skin does not feel tight (100%)
   The skin is perfectly cleansed (95.65%)
   The product is suitable for your skin type (95.65%)
   Your impression of the product is good (95.65%)

Under the conditions of the study, the product exhibited:
excellent dermatological tolerance, according to the scale adopted, on oily facial skin;
and very good cosmetic acceptability, with 96% favorable opinions.

Hibiscus Flower Acid Cleanser
Items relating to the efficacy of the product (positive responses≥75%, threshold of statistical significance for 22 volunteers analysed)
   The product perfectly cleanses the skin (100%)
   The product is pleasant to use (95.45%)
   The product is easy to apply (95.45%)
   The product is easy to rinse off (100%)
   The skin is soft (100%)
   The skin is clearer (100%)
   The product removes impurities (100%)
   The skin is clean and healthy (100%)
   The product improves the overall appearance and condition of the skin (100%)
   The product reduces the feeling of tightness and provides a feeling of comfort (100%)
   The skin does not feel tight (100%)
   The product is suitable for your skin type (100%)
   The complexion is brighter (100%)
   Pores appear tightened (100%)
   The skin is smooth (100%)
   The product is gentle on the skin (100%)
   The skin is protected from external aggressions (100%)
   The complexion is even (100%)
   The skin is perfectly cleansed (100%)
   Your impression of the product is good (100%)

Under the conditions of the study, the product exhibited:
excellent dermatological tolerance, according to the scale adopted, on normal facial skin;
excellent cosmetic acceptability, with 100% favorable opinions.

Triple Oil Cleanser
Items relating to the efficacy of the product (positive responses≥75%, threshold of statistical significance for 23 volunteers analysed)
   The product perfectly cleanses the skin (100%)
   The product is pleasant to use (100%)
   The product is easy to apply (100%)
   The product is easy to rinse off (100%)
   The skin is soft (100%)
   The skin is clearer (100%)
   The product removes impurities (100%)
   The skin is clean and healthy (100%)
   The product improves the overall appearance and condition of the skin (100%)
   The product reduces the feeling of tightness and provides a feeling of comfort (100%)
   The product does not dry out the skin (100%)
   The product is suitable for your skin type (100%)
   The skin is nourished (100%)
   The skin is smooth (100%)
   The skin does not feel tight (100%)
   The product is gentle on the skin (100%)
   The skin is protected from external aggressions (100%)
   The skin is perfectly cleansed (100%)
   Your impression on the product is good 100%)

Under the conditions of the study, the product exhibited:
excellent dermatological tolerance, according to the scale adopted, on normal or dry facial skin;
and excellent cosmetic acceptability, with 100% favorable opinions.

1.-38. (canceled)

39. A combination consisting of the following 5 constituents:
a powder adhesion agent,
mannitol,
α-glucan oligosaccharide,
at least one oil, and
at least one surfactant,
said combination being in powder form,
in which the mass percentage of water is between 0 and 10% relative to the total mass of the combination,
said adhesion agent, said mannitol, said α-glucan oligosaccharide, and said at least one surfactant being in powder form, said at least one oil being in particular in powder form,
in particular in which the density of the powder is comprised from 0.35 to 0.55 g/ml, in particular from 0.4 to 0.5 g/ml.

40. The combination according to claim 39, free of mannitol.

41. The combination according to claim 39, in which:
the powder adhesion agent is chosen from: corn starch, acacia powder, maltodextrin powder, apple extract powder, tapioca powder, rice powder, rice bran extract, and arrowroot powder or a mixture of these,
and/or in which the at least one oil is chosen from: jojoba oil, sweet almond oil, bitter almond oil, Argan oil, sesame oil, camellia oil, safflower oil, moringa oil, Castor oil, coconut oil, apricot oil, linseed oil, pomegranate oil, grapeseed oil, prickly pear oils, marula oil, evening primrose oil, avocado oil, raspberry oil, borage oil, olive oil, neem oil, sunflower oil, walnut or hazelnut oil, amla oil, plum oil, coffee oil, hemp oil, a vanilla macerate, an oil powder, and a fermented oil, or a mixture of these,
and/or in which the surfactant is a surfactant chosen from: lauryl glucoside, decyl glucoside, caprylyl/capryl glucoside, coco-glucoside, hexyl glucoside, sodium cocoyl glutamate, disodium cocoyl glutamate, sodium stearoyl glutamate, sodium lauroyl glutamate, sodium myristoyl glutamate, coco-betaine, cocamidopropyl betaine, disodium cocoamphodiacetate, sodium arganamphoacetate, sodium babassuamphoacetate, sodium cocoabutteramphoacetate, sodium cottonseedamphoacetate, sodium sheabutteramphoacetate, sodium mangoamphoacetate, sodium olivamphoacetate, sodium ricebranamphoacetate, sodium sweetalmondamphoacetate, sodium sunflowerseedamphoacetate, potassium cocoate, potassium palmate, potassium laurate, potassium olivate, sodium coco-sulfate, sodium cocoamphoacetate, disodium coco-glucoside citrate, sodium coco-glucoside tartrate, sodium lauroamphoacetate, cocoyl methyl glucamide, capryloyl/caproyl methyl glucamide, lauroyl/myristoyl methyl glucamide, ammonium lauryl sulfate, ammonium coco-sulfate, magnesium lauryl sulfate, sodium C12-18 alkyl sulfate, zinc coco-sulfate, sodium cocoyl alaninate, sodium lauryl sulfate, sodium cetearyl sulfate, sodium cocoyl isethionate, sodium methyl oleoyl taurate, sodium methyl cocoyl taurate, cocamide MEA, cocamide MIPA, sodium C14-16 olefin sulfonate, sodium lauryl sulfoacetate, sodium lauroyl sarcosinate, sodium laureth sulfate, ammonium laureth sulfate, cocamidopropyl hydroxysultaine, disodium laureth sulfosuccinate, disodium lauryl sulfosuccinate, sodium myreth sulfate, laureth-5 carboxylic acid, and saponin,
in particular, in which the surfactant is an anionic surfactant chosen in particular from: sodium cocoyl glutamate, and sodium cocoyl isethionate, or a mixture of these.

42. The combination according to claim 39, in which:
the total quantity of powder adhesion agent is comprised from 5 to 85%, in particular from 5 to 15%, or from 70 to 85%, in percentage by weight relative to the total weight of the combination.
and/or in which the total quantity of mannitol is comprised from 5 to 20%, in particular from 5 to 15% in percentage by weight relative to the total weight of the combination,
and/or in which the total quantity of α-glucan oligosaccharide is comprised from 0.5 and 2%, in particular approximately 1% in percentage by weight relative to the weight total of the combination,
and/or in which the total quantity of oil is comprised from 1 to 50%, in particular from 1 to 10%, in particular from 2 to 8% in percentage by weight with respect to the total weight of the combination,
and/or in which the total quantity of surfactant is comprised from 1 and 80%, in particular from 2 to 20%, or from 40 to 80% in percentage by weight relative to the total weight of the combination.

43. The combination according to claim 39, said combination being in the form:
of a shampoo, said shampoo comprising in particular:
two surfactants in a total amount comprised from 40 to 80%, in particular from 70 to 80%, as a percentage by weight relative to the total weight of the composition, and a powder adhesion agent, in an amount comprised from 5 to 15% in percentage by weight relative to the total weight of the composition, or
of a cleaner, said cleaner comprising in particular:
a surfactant in an amount comprised from 2 to 20%, in particular from 5 to 10% by weight percentage relative to the total weight of the composition, and a powder adhesion agent, in an amount of 70 to 85% in percentage by weight relative to the total weight of the composition.

44. A cosmetic composition, comprising a combination according to claim 39, in a cosmetically acceptable medium,
said composition being in the form of a powder,
in which the mass percentage of water is comprised from 0 to 10% relative to the total mass of the composition,
in particular in which the density of the powder is comprised from 0.35 to 0.55 g/ml, in particular from 0.4 to 0.5 g/ml.

45. A cosmetic composition, comprising a combination according to claim 40, in a cosmetically acceptable medium,
said composition being in the form of a powder,
in which the mass percentage of water is comprised from 0 to 10% relative to the total mass of the composition,
wherein said composition further comprises a pigment or piroctone olamine,
in particular in which the density of the powder is comprised from 0.35 to 0.55 g/ml, in particular from 0.4 to 0.5 g/ml.

46. The cosmetic composition according to claim 44, said combination being present at a rate of 70 to 100%, in particular 85 to 100% in percentage by weight relative to the total weight of the composition.

47. The cosmetic composition according to claim 44, in which the total quantity of powder adhesion agent is comprised from 3.5 to 85%, in particular from 3.5 to 15%, or from 50 to 85%, as a percentage by weight relative to the total weight of the composition,
and/or in which the total quantity of mannitol is comprised from 3.5 to 20%, in particular from 8 to 15%, as a percentage by weight relative to the total weight of the composition,
and/or in which the total quantity of α-glucan oligosaccharide is comprised from 0.35 to 2%, in particular approximately 1%, as a percentage by weight relative to the total weight of the composition,
and/or in which the total quantity of oil is comprised from 0.7 to 50% in percentage by weight relative to the total weight of the composition,
and/or in which the total amount of surfactant is comprised from 0.7 to 80%, in particular comprised from 1.4 to 20%, or comprised from 30 to 80% as a percentage by weight relative to the total weight of the composition.

48. The cosmetic composition according to claim 44, in which
the composition also comprises at least one ingredient, in particular an active principle, in particular chosen from: flower acid, Zinc PCA, a pigment, the coal, an antioxidant, in particular vitamin C, or one of its derivatives, powdered tea, including matcha green tea, fermented tea, powdered kombucha tea, centella asiatica, or madecassoside, sugars, in particular xylitol, rhamnose, or fructooligosaccharides, the aloe vera, a protein, in particular pea protein, soy protein, wheat protein, or rice protein, sweet almond milk, or one or more of its components, in particular prunus dulcis, rice syrup or acacia fibre, a perfume, in particular an apricot almond perfume, ghassoul, white clay, especially kaolin, sodium hyaluronate, hyaluronic acid, lactic acid, vanilla macerate, maltodextrin, bamboo powder, spirulina, phycocyanin, fermented mushrooms, guarana, caffeine, acerola, chamomile, Ayurvedic powders, vitamins, in particular vitamin B3, or vitamin B5, glycosphingolipids and glycolipids, exopolysaccharides, and postbiotics, in particular lactobacillus ferment, pyroctolamine, piroctone olamine, an extract of camellia seeds, corn flower extract, chamomile flower extract, or a mixture of these, said at least one ingredient is in particular solid at room temperature.

49. The cosmetic composition according to claim 44, said composition being in the form:
of a shampoo, said shampoo comprising in particular:
two surfactants in a total amount comprised from 30 to 80%, in particular from 50 to 80% as a percentage by weight relative to the total weight of the composition, and a powder adhesion agent, in an amount comprised from 3.5 to 15% in percentage by weight relative to the total weight of the composition, or
of a cleaner, said cleaner comprising in particular:
a surfactant at a rate of 1.4 to 20% in percentage by weight relative to the total weight of the composition, and a powder adhesion agent, in an amount comprised from 50 to 85%, in particular from 70 to 85% in percentage by weight relative to the total weight of the composition.

50. The cosmetic composition according to claim 44, in which said composition comprises, in percentage by weight relative to the total weight of the composition:
a powder adhesion agent, in particular corn starch, at a rate of 8 to 11%, and
mannitol at a rate of 9 to 11%,
α-glucan oligosaccharide, at a rate of 0.5 to 1.5%,
Argan oil, at a rate of 3 to 5%,
sodium cocoyl glutamate at a rate of 25 to 35%,
sodium cocoyl isethionate at a rate of 35 to 45%,
aloe vera at a rate of 0.5 to 1%,
pea protein at 2 to 3%, and
apricot almond flavor at a rate of 1 to 3%,
said composition being in particular a shampoo.

51. The cosmetic composition according to claim 44, in which said composition comprises, in percentage by weight relative to the total weight of the composition:
a powder adhesion agent, in particular corn starch, at a rate of 10 to 12%,
mannitol at a rate of 9 to 10%,
α-glucan oligosaccharide, at a rate of 0.5 to 1.5%,
sweet almond milk, at a rate of 6 to 7%,
sodium cocoyl glutamate at a rate of 25 to 35%, and
sodium cocoyl isethionate at a rate of 35 to 45%,
said composition being in particular a shampoo.

52. The cosmetic composition according to claim 44, in which said composition comprises, in percentage by weight relative to the total weight of the composition:
a powder adhesion agent, in particular corn starch, at a rate of 8 to 11%,
mannitol at a rate of 7 to 9%,
α-glucan oligosaccharide, at a rate of 0.5 to 1.5%,
Ghassoul, at a rate of 4 to 6%,
sodium cocoyl glutamate at a rate of 25 to 35%,
sodium cocoyl isethionate at a rate of 35 to 45%,
bamboo powder at a rate of 2 to 4%,
jojoba oil at a rate of 1 to 3%, and
herbal and tea perfume at a rate of 1 to 3%,
said composition being in particular a shampoo.

53. The cosmetic composition according to claim 45, in which said composition comprises, in percentage by weight relative to the total weight of the composition:
sodium cocoyl glutamate at a rate of 25 to 35%,
sodium cocoyl isethionate at a rate of 35 to 45%,
a powder adhesion agent, in particular corn starch,
α-glucan oligosaccharide, at a rate of 0.5 to 1.5%,
sunflower oil,
chamomile flower extract,
piroctone olamine
flower acid, and
herbal and tea fragrance,
said composition being in particular a shampoo.

54. The cosmetic composition according to claim 45, in which said composition comprises, in percentage by weight relative to the total weight of the composition:
sodium cocoyl glutamate at a rate of 25 to 35%,
sodium cocoyl isethionate at a rate of 35 to 45%,
a powder adhesion agent, in particular corn starch,
α-glucan oligosaccharide, at a rate of 0.5 to 1.5%,
argan oil,
rice protein,
pea protein,
benzene sulfonic acid 2-[(9,10-dihydro-4-hydroxy-9,10-dioxo-1-anthracenyl) amino]-5-methyl sodium monosalt,
erythrol, and
herbal and tea fragrance,
said composition being in particular a shampoo.

55. The cosmetic composition according to claim 44, in which said composition comprises, in percentage by weight relative to the total weight of the composition:
a powder adhesion agent, in particular corn starch, at a rate of 75 to 79%,
mannitol at a rate of 12 to 14%,
α-glucan oligosaccharide, at a rate of 0.5 to 1.5%,
sodium cocoyl glutamate at a rate of 4 to 6%,
sesame oil at a rate of 0.5 to 2%,
jojoba oil at a rate of 0.5 to 2%,
zinc PCA at a rate of 0.5 to 2%, and
coal at a rate of 0.5 to 2%,
said composition being in particular a cleanser.

56. The cosmetic composition according to claim 44, in which said composition comprises, in percentage by weight relative to the total weight of the composition:
a powder adhesion agent, in particular corn starch, at a rate of 72 to 76%,
mannitol at a rate of 9 to 11%,
α-glucan oligosaccharide, at a rate of 0.5 to 1.5%,
sodium cocoyl glutamate at a rate of 4 to 6%,
sweet almond oil at a rate of 2 to 4%,
ascorbyl glucoside at a rate of 2 to 4%,
jojoba oil at a rate of 2 to 4%, and
flower acid at a rate of 1 to 3%,
said composition being in particular a cleanser.

57. The cosmetic composition according to claim 44, in which said composition comprises, in percentage by weight relative to the total weight of the composition:
a powder adhesion agent, in particular corn starch, at a rate of 65 to 75%,
mannitol at a rate of 14 to 16%,
α-glucan oligosaccharide, at a rate of 0.5 to 1.5%,
sodium cocoyl glutamate at a rate of 4 to 6%,
safflower oil at a rate of 2 to 4%,
jojoba oil at a rate of 2 to 4%, and
spirulina at a rate of 2 to 4%,
said composition being in particular a cleanser.

58. The cosmetic composition according to claim 44, in which said composition comprises, in percentage by weight relative to the total weight of the composition:
a powder adhesion agent, in particular corn starch, at a rate of 70 to 76%,
mannitol at a rate of 13 to 17%,
α-glucan oligosaccharide, at a rate of 0.5 to 1.5%,
sodium cocoyl glutamate at a rate of 4 to 6%,
safflower oil at a rate of 1 to 3%,
argan oil at a rate of 1 to 3%, and
hemp oil at a rate of 1 to 3%.
said composition being in particular a cleanser.
